(19) United States
(12) Patent Application Publication
(10) Pub. No.: US 2003/0158443 A1
(43) Pub. Date: Aug. 21, 2003

(54) Combinatorial active compound synthesis and its intermediates

(76) Inventors: Klaus Haaf, Kelkeim (DE); Lothar Willms, Hofheim (DE)

Correspondence Address: FROMMER LAWRENCE & HAUG, 745 FIFTH AVENUE- 10TH FL., NEW YORK, NY, 10151, US

(21) Appl. No.: 10/263,404

(22) Filed: Oct. 2, 2002

Related U.S. Application Data
(63) Continuation of application No. 08/948,557, filed on Oct. 10, 1997, now abandoned.

(30) Foreign Application Priority Data
Oct. 15, 1996 (DE) 19642429.1

(51) Int. Cl. C07C 313/28

(52) U.S. Cl. 564/91; 558/413; 560/12

(57) ABSTRACT

The invention relates to a resin-bound synthesis process for the preparation of chemical compounds of the formula (I)

R¹—SO₂—NH—CO—R² (I)

in which R¹ and R² each are an organic radical, which comprises

a) reacting a resin-linker compound of the formula [resin polymer]-[L-Nuc]ₙ (II) in which nucleofugic groups Nuc are bonded to a resin [=resin polymer] via the linker L, with an acylsulfonamide of the formula E¹-SO₂—NH—CO-E² (III) in which E¹ and E² independently of one another in each case are an organic radical suitable for the preparation of the radicals R¹ and R² in compound (I),

in the presence of a condensing agent to give a resin-bound adduct of the formula

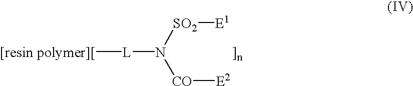

in which [resin polymer], L, n, E¹ and E² are as defined in formula (II) or formula (III),

b) derivatizing the adduct (IV) obtained in one or more further reaction steps on the organic radicals E¹ or E² to give the radicals R¹ or R², and

c) cleaving the compound of the formula (I) from the resin-linker adduct of the formula (IV″) obtained.

The invention relates to the technical field of the synthesis of active compounds having certain common structural features.

The increasing demands on the properties of novel biologically active substances for plant protection or medicine mean that the development of an active compound which is ready for marketing is associated with the preparation and testing of increasingly large numbers of test substances. In the estimation of many specialists, this tendency will probably persist despite improved knowledge about the biochemistry of known active compounds and computer-assisted calculations of molecular structures and properties (“molecular modeling”). In order not to allow the expense and consumption of time to increase equally, the object for research into novel active compounds is to develop more effective methods for the preparation of large numbers of novel test compounds.

The methods for the systematic preparation of large numbers of test compounds and especially methods suitable for their analysis are summarized under the term “combinatorial chemistry”; cf., for example, J. S. Früchtel, G. Jung in Angew. Chem. 108 (1996) pp. 19 ff.

Some combinatorial synthesis methods are aimed at preparing jointly (“in a pool”), in a manner which is as effective and standardized as possible, a large number of structurally variant compounds in as few reaction steps as possible and jointly testing them for biological action; cf. for example the divide, couple and recombine method according to a) K. S. Lam, S. E. Salmon, E. M. Hersh, V. J. Hruby, W. M. Kazmeiersky, R. J. Knapp in Nature 82 (1991) 354, b) A. Furka, F. Sebestyen, M. Asgedom, G. Dibo in Int. J. Pept. Protein Res. 37 (1991) 487. If an entire group of compounds (“pool”) contains no active compound, a single joint test suffices to exclude these structural variants. If the joint test, however, indicates activity, the variation in the preparation of the test compounds can be decreased in a controlled manner in order to limit the group containing the active compound or the active compounds and finally to determine the structure of the active compounds. As a rule, however, the method described can no longer be used efficiently when it concerns the optimization of active compound structures and many similarly active compounds are present in the group of test compounds or alternatively when larger amounts of the compounds are needed for the first tests.

To achieve the last-mentioned object, the starting compound used is often a compound with known biological action, the so-called lead structure or lead compound, and the structure of the lead compound is varied systematically with the aid of a preparation process which is standardized to a great extent, by use of a large number of different starting materials. The individual compounds prepared in each case are then tested individually for their biological action in order to find the optimally active compound with the same type of action.

The known synthesis methods from combinatorial chemistry (see J. S. Früchtel, G. Jung in Angew. Chem. 108 (1996) p. 19 ff. and references cited there) include a group of methods in which the respective active compound is prepared stepwise bound to a solid, in particular bound to a synthetic or natural resin. With the aid of the binding to the solid, e.g. to the resin in the form of particles of large particle size or spheres, the intermediates are in principle handleable macroscopically. The synthesis of an active compound via several reaction steps then needs less expenditure on isolation and purification than in conventional methods, because these steps as a rule can be effected in the form of a simple filtration and washing of the resinous substances. The resin-bound finished active compound molecule must finally be removed again from the resin.

In the choice of suitable resin-molecule systems, problems fundamentally result due to the conflict of aims in guaranteeing both a desired high stability of the bond between entities synthesized and the resin when using different reaction types and conditions and in making possible a gentle method for the predominant or complete removal of the finished synthesis product from the resin.

The invention is based on the object of making available a combinatorial synthesis method based on resin-bound synthesis components and products, which allows the synthesis of a wide variation of biologically active compounds of identical partial structure.

The invention relates to a process for the preparation of chemical compounds of the formula (I)

R¹—SO₂—NH—CO—R² (I)

in which R¹ and R² are each an organic radical,

which comprises

a) reacting a resin-linker compound of the formula (II)

[resin polymer]-[L-Nuc]ₙ (II)

in which

[resin polymer] is the radical of a resin which is connected via n binding sites with the n groups of the formula -L-Nuc defined in formula (II),

L is in each case an organic linker,

Nuc is a nucleofugic group (leaving group) or a group to be activated under the reaction conditions to give a leaving group, e.g. OH, amino, halogen, mesyl or tosyl,

n is the number of functional groups L-Nuc on the resin, which depends on the molecular weight of the resin polymer and is greater than or equal to 1,

with an acylsulfonamide of the formula (III)

E¹-SO₂—NH—CO-E² (III)

in which E¹ and E² independently of one another in each case are an organic radical which is suitable for the preparation of the radicals R¹ and R² in compound (I),

in the presence of a condensing agent to give a resin-bound adduct of the formula (IV)

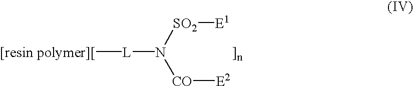

in which [resin polymer], L, n, E¹ and E² are as defined in formula (II) or formula (III),

b) derivatizing the adduct (IV) obtained in one or more further reaction steps on the organic radicals E¹ or E² and thus optionally reacting via resin-bound intermediates of the formula (IV′), which in contrast to formula (IV) contain the organic radicals (E¹)′ or (E²)′ of the derivatives, to give the compound (IV″)

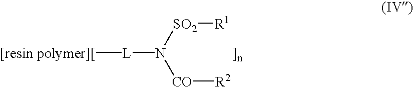

in which R¹ and R² are as defined in formula (I) and [resin polymer], L and n are as defined in formula (II) or formula (IV), and

c) removing the compound of the formula (I) from the resin-linker adduct of the formula (IV″).

The invention also relates to the individual steps of the process according to the invention and the novel adducts of the formula (IV) and also of the formula (IV′) and (IV″). The latter two compound groups are likewise compounds of the formula (IV), in which, however, the radicals E¹ and E² have the meaning of the appropriately modified organic radicals (E¹)′ or (E²)′ and R¹ or R², respectively, of the derivatized intermediates.

One aspect of the invention is the binding carried out in step a) of compounds with the partial structure —SO₂—NH—CO— (acylsulfonamide) to a resinous substance (resin polymer). The method of binding at the same time also makes possible a method for the extensive or complete removal of the compounds with retention of the partial structure —SO₂—NH—CO— [see step c)], it being possible between binding and removal of the resinous substance for structural modifications to be performed in other parts of the molecule. The particular conditions are fulfilled by the function of the linker, whose structure fixes the type of reaction for the chemical binding and removal of the compound having the partial structure mentioned. A method of this type for the binding of compounds having the partial structure —SO₂—NH—CO— to a resinous substance was hitherto unknown. Among the large number of known types of reaction which are possible in principle for the reaction of an acylsulfonamide at its amido group, such as, for example, N-alkylation, N-acylation, addition to alkenes and activated alkenes etc., there is, however, hardly a suitable method which makes possible removal with retention of the acylsulfonamide structure. Many of the reactions require conditions which are incompatible with the resinous substance, e.g. temperatures which are too high or too low, strongly basic or acidic conditions. The resinous substance must be swellable in the reactions and must remain largely unchanged in its polymer structure, because it has to be employed over several reaction steps and has to be removed again after the reaction or reaction sequence.

According to the invention, however, the acylsulfonamides can surprisingly bond to the resin polymer in a suitable manner using a number of linkers whose employability was not to be assumed beforehand for the abovementioned reasons and which now open up further analogous possibilities (for explanations see below).

The invention therefore relates in particular to the reaction stages a) (binding) and c) (removal) and also to the resin-linker adducts (IV), (IV′) and (IV″). The derivatization reaction or reactions in stage b) (derivatization) are as a rule of the type known and can mostly be used under analogous reaction conditions, some preferred process measures being limited by particular features of the resinous substance. In the choice of the derivatization reactions, possible interactions between functional groups in the organic radicals E¹ and E² or (E¹)′ and (E²)′ or R¹ and R² are to be taken into consideration in the manner known to the person skilled in the art. A general restriction of the structure of the organic radicals in the compounds (I), (IV), (IV′) and (IV″) does not exist.

In the formula (I) and the other formulae (III), (IV), (IV′), (IV″) etc., an organic radical R¹ or R² is a carbon-containing radical, for example an optionally substituted (hetero)aromatic radical or an aliphatic, i.e. nonaromatic, organic radical which, apart from carbon atoms and hydrogen atoms, can also contain heteroatoms and/or can be substituted and which can also be connected to other parts of the molecule via heteroatoms, e.g. in the case of the compounds of the formula (I) can be connected via heteroatoms to the SO₂ group or the carbonyl group. The suitable organic radicals can be very different in size; an organic radical including possibly contained substituents preferably contains less than 30 carbon atoms, in particular 1 to 20 carbon atoms, smaller radicals having 1 to 12 carbon atoms as a rule being preferred.

Possible substituents of an organic radical are likewise (hetero)aromatic and aliphatic radicals, including functional groups, the functional groups preferably being highly compatible with the functional groups otherwise present in the compound of the formulae (I) and (II). For example, as functional groups, no oxidative groups should be present if the linker is sensitive to oxidation and thus would react even under the conditions of the combinatorial synthesis.

Organic radicals are, for example, optionally substituted hydrocarbon radicals or hydrocarbon-oxy radicals. A hydrocarbon radical is a straight-chain, branched or cyclic and saturated or unsaturated aliphatic or aromatic hydrocarbon radical, e.g. alkyl, alkenyl, alkynyl, cycloalkyl, cycloalkenyl or aryl; a hydrocarbon radical is preferably alkyl, alkenyl or alkynyl having up to 12 carbon atoms or cycloalkyl having 3, 4, 5, 6, 7 or 8 ring atoms or aryl; the same applies to a hydrocarbon radical in a hydrocarbon-oxy radical.

Aryl is a mono-, bi or polycyclic, carbocyclic aromatic ring system; in the substituted case, or more precisely in the cyclically substituted case, bicyclic or polycyclic ring systems having at least one aromatic ring which is fused to one or more cycloaliphatic, optionally partially unsaturated rings, are in particular also included. Optionally cyclically substituted aryl is, for example, phenyl, naphthyl, tetrahydronaphthyl, indenyl, indanyl, pentalenyl, fluorenyl and the like, it being possible for the ring systems mentioned to additionally be further substituted in the generally substituted case; preferably aryl is an unsubstituted phenyl or naphthyl ring; substituted aryl is preferably a phenyl radical which is unsubstituted or substituted, the substituents not being fused rings.

Heteroaryl or a heteroaromatic radical is a mono-, bi- or polycyclic aromatic ring system in which at least 1 ring contains one or more heteroatoms, for example pyridyl, pyrimidinyl, pyridazinyl, pyrazinyl, triazinyl, thienyl, thiazolyl, oxazolyl, isoxazolyl, furyl, pyrrolyl, pyrazolyl and imidazolyl. In the substituted case, bicyclic or polycyclic aromatic, benzo-fused compounds or compounds fused to cycloaliphatic rings, e.g. quinolinyl, benzoxazolyl etc., are in particular also included. Heteroaryl also includes a heteroaromatic ring which is preferably 5- or 6-membered and contains 1, 2 or 3 heterocyclic ring atoms, in particular from the group consisting of N, O and S.

A heterocyclic radical (heterocyclyl) or ring (heterocycle) can be saturated, unsaturated or heteroaromatic (heteroaryl); it contains one or more heterocyclic ring atoms, preferably from the group consisting of N, O and S; it is preferably a nonaromatic ring having 3 to 8 ring atoms and 1 to 3 heterocyclic ring atoms from the group consisting of N, O and S or is a heteroaromatic ring having 5 or 6 ring atoms and contains 1, 2 or 3 heterocyclic ring atoms from the group consisting of N, 0 and S. The radical can be, for example, a heteroaromatic radical or ring as defined above or is a partially hydrogenated radical such as oxiranyl, pyrrolidyl, piperidyl, piperazinyl, dioxolanyl, morpholinyl or tetrahydrofuryl. Possible substituents for a substituted heterocyclic radical are the substituents mentioned further below, additionally also oxo. The oxo group can also occur on the heterocyclic ring atoms, which can exist in various oxidation states, e.g. with N and S.

Substituted radicals, such as substituted hydrocarbons, e.g. substituted alkyl, alkenyl, alkynyl, aryl, phenyl and benzyl, or substituted heteroaryl, a substituted bicyclic radical or ring or a substituted bicyclic radical, optionally with aromatic components, are, for example, a substituted radical derived from the unsubstituted parent substance, these substituents being, for example, one or more, preferably 1, 2 or 3 radicals from the group consisting of halogen, alkoxy, haloalkoxy, alkylthio, hydroxyl, amino, nitro, cyano, azido, alkoxycarbonyl, alkylcarbonyl, formyl, carbamoyl, mono- and dialkylaminocarbonyl, substituted amino such as acylamino, mono- or dialkylamino, and alkylsulfinyl, haloalkylsulfinyl, alkylsulfonyl, haloalkylsulfonyl and, in the case of cyclic radicals, also alkyl and haloalkyl as well as unsaturated aliphatic radicals corresponding to the saturated hydrocarbon-containing radicals mentioned, such as alkenyl, alkynyl, alkenyloxy, alkynyloxy etc. In the case of radicals having carbon atoms, those having 1 to 4 carbon atoms, in particular 1 or 2 carbon atoms, are preferred. As a rule, preferred substituents are those from the group consisting of halogen, e.g. fluorine and chlorine, C₁-C₄-alkyl, preferably methyl or ethyl, C₁-C₄-haloalkyl, preferably trifluoromethyl, C₁-C₄-alkoxy, preferably methoxy or ethoxy, C₁-C₄-haloalkoxy, nitro and cyano. The substituents methyl, methoxy and chlorine are particularly preferred here.

Optionally substituted phenyl is preferably phenyl which is unsubstituted or mono- or polysubstituted, preferably up to trisubstituted, by identical or different radicals from the group consisting of halogen, C₁-C₄-alkyl, C₁-C₄-alkoxy, C₁-C₄-haloalkyl, C₁-C₄-haloalkoxy and nitro, e.g. o-, m- and p-tolyl, dimethylphenyls, 2-, 3- and 4-chlorophenyl, 2-, 3- and 4-trifluoro- and -trichlorophenyl, 2,4-, 3,5-, 2,5- and 2,3-dichlorophenyl, o-, m- and p-methoxyphenyl.

The radicals alkyl, alkoxy, haloalkyl, haloalkoxy, alkylamino and alkylthio as well as the corresponding unsaturated and/or substituted radicals are in each case straight-chain or branched in the carbon structure. If not specially stated, in these radicals the lower hydrocarbon structures, e.g. having 1 to 4 carbon atoms or, in the case of unsaturated groups, having 2 to 4 carbon atoms, are preferred. Alkyl radicals, even in the associated meanings such as alkoxy, haloalkyl etc., are, for example, methyl, ethyl, n- or i-propyl, n-, i-, t- or 2-butyl, pentyls, hexyls, such as n-hexyl, i-hexyl and 1,3-dimethylbutyl, heptyls, such as n-heptyls, 1-methylhexyl and 1,4-dimethylpentyl. Cycloalkyl is a cycloaliphatic hydrocarbon radical such as cyclopropyl, cyclobutyl, cyclopentyl or cyclohexyl etc.; alkenyl, cycloalkenyl and alkynyl have the meaning of the possible unsaturated radicals corresponding to the alkyl radicals; alkenyl is, for example, allyl, 1-methyl-prop-2-en-1-yl, 2-methylprop-2-en-1-yl, but-2-en-1-yl, but-3-en-1-yl, methyl-but-3-en-1-yl and 1-methylbut-2-en-1-yl; cycloalkenyl is, for example, cyclopentenyl and cyclohexenyl; alkynyl is, for example, propargyl, but-2-yn-1-yl, but-3-yn-1-yl, 1-methylbut-3-yn-1-yl. Alkenyl in the form “(C₃-C₄)alkenyl” or “(C₃-C₆)alkenyl” is preferably an alkenyl radical having 3 to 4 or 3 to 6 carbon atoms, in which the double bond is not on the carbon atom which is connected to the other part of the compound molecule (“yl” position). The same applies to (C₃-C₄)alkynyl etc.

Halogen is, for example, fluorine, chlorine, bromine or iodine, haloalkyl, -alkenyl and -alkynyl are alkyl, alkenyl or alkynyl, which is partly or completely substituted by halogen, preferably by fluorine, chlorine and/or bromine, in particular by fluorine or chlorine, e.g. CF₃, CHF₂, CH₂F, CF₃CF₂, CH₂FCHCl₂, CCl₃, CHCl₂, CH₂CH₂Cl; haloalkyl is, for example, OCF₃, OCHF₂, OCH₂F, CF₃CF₂O, OCH₂CF₃ and OCH₂CH₂Cl; the same applies to haloalkenyl and other radicals substituted by halogen.

Mono- or disubstituted amino is a chemically stable radical from the group consisting of the substituted amino radicals which are N-substituted, for example, by one or two identical or different radicals from the group consisting of alkyl, alkoxy, acyl and aryl; preferably monoalkylamino, dialkylamino, acylamino, arylamino, N-alkyl-N-arylamino as well as N-heterocycles; in this case alkyl radicals having 1 to 4 carbon atoms are preferred; aryl is in this case preferably phenyl or substituted phenyl; for acyl the definition mentioned further below applies, preferably (C₁-C₄)-alkanoyl. The same applies to substituted hydroxylamino or hydrazino.

An acyl radical is the radical of an organic acid, e.g. the radical of a carboxylic acid and radicals of acids derived therefrom such as the thiocarboxylic acid, optionally N-substituted iminocarboxylic acids, or the radical of carboxylic acid monoesters, optionally N-substituted carbamic acid, sulfonic acids, sulfinic acids, phosphonic acids, phosphinic acids. Acyl is, for example, formyl, alkylcarbonyl such as (C₁-C₄-alkyl)carbonyl, phenylcarbonyl, where the phenyl ring can be substituted, for example, as shown above for phenyl, or alkyloxycarbonyl, phenyloxycarbonyl, benzyloxycarbonyl, alkylsulfonyl, alkylsulfinyl, N-alkyl-1-iminoalkyl and other radicals of organic acids.

The formulae also include stereoisomers which contain, for example, one or more asymmetric carbon atoms or alternatively double bonds which are not separately indicated in the respective formula. The possible stereoisomers defined by their specific spatial form with identical chemical linkage, such as enantiomers, diastereomers, Z and E isomers are thus all included by the formula and can be obtained from mixtures of the stereoisomers by customary methods or alternatively can be prepared by stereoselective reactions in combination with the use of stereochemically pure starting substances.

The formulae also include tautomers of the compounds described, if they are formed by proton migration and if they are chemically stable tautomers.

The compounds of the formula (I) can form salts in which the hydrogen of the —SO₂—NH group or alternatively other acidic hydrogen atoms (e.g. from COOH, inter alia) is replaced by a cation suitable for agriculture. These salts are, for example, metal salts; preferably alkali metal or alkaline earth metal salts, in particular sodium and potassium salts, or alternatively ammonium salts or salts with organic amines. Salt formation can also take place by addition of an acid to basic groups, such as, for example, amino and alkylamino. Suitable acids for this purpose are strong inorganic and organic acids, for example HCl, HBr, H₂SO₄ or HNO₃.

The organic linker L in the compounds of the formulae (II), (IV), (IV′) and (IV″) has the function of a bridge between the resin polymer and the part of the molecule with the sulfonamidocarbonyl group from the compound of the formula (III) or formula (I).

The linker must make possible the binding of the part of the molecule mentioned and its later removal. The linker must additionally be able to be applied to the resin polymer, to be specific as a rule by means of a chemical reaction if the resin polymer cannot already be synthesized from suitable monomers which contain the linker.

Suitable linkers L are radicals which are structurally very different, which must have suitable binding sites and functional groups depending on the binding sites on the resin polymer. Surprisingly, according to the invention very many linkers appear to be suitable which can also be employed in resin-bound synthesis for the binding of carboxylic acids, for example of amino acids, in peptide synthesis.

Compounds (linker components) which can be employed for the synthesis of the linker in combination with a resin containing amino groups, e.g. an aminomethylenepolystyrene resin, or a resin containing hydroxyl groups, are linker components having a carboxylic acid group. The preparation of the resin-linker compound of the formula (II) is then carried out in each case by reaction of the carboxyl group of a linker component with an amino group or hydroxyl group of the resin (amide formation or ester formation).

In some cases, the linkers are also prepared stepwise; in a first step a carboxylic acid is condensed with the resin containing amino groups and the modified resin obtained is further modified on the introduced groups as far as the desired resin-linker compound.

In addition, resin-linker compounds are known which are synthesized on the basis of further resins and in another manner.

Examples of linker components and resin-linker compounds of the formula (II) are shown below in Table 1; the linker component is in each case the compound of the formula (V)

Z-L-Nuc (V),

in which Z is the leaving group or functional group to be activated to give the leaving group, which is replaced in the reaction with the amino group or hydroxyl group of the resin; in the case in which the resin-linker compound (II) is prepared differently or the preparation is not given in detail, the radical Z=“Polymer” is given, which indicates the binding site of the functional group -L-Nuc on the resin polymer:

TABLE 1
Linker components and/or resin-linker compounds

| Linker components Z—L—Nuc or | |
|---|---|
| functional group on the polymer | Reference |
| 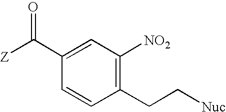 | F. Albericio, E. Giralt, R. Eritja, Tetrahedron Lett. 1991, 1515 |
| Z = OH | |
| Nuc = OH | |
| Z—CO—CH₂—SO₂—CH₂CH₂—Nuc | S. B. Katti, P. K. Mirsa, |
| Z = OH | W. Hag, K. B. Mathur, |
| Nuc = OH | J. Chem. Soc. Chem. |
| | Commun. 1992, 843 |
| 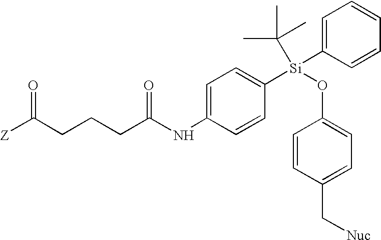 | D. G. Mullen, G. Barany, J. Org. Chem. 53 (1988) 5240 |
| Z = OH | |
| Nuc = OH | |
| 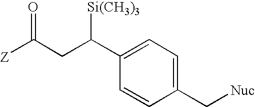 | D. G. Mullen, G. Barany, J. Org. Chem. 53 (1988) 5240; R. Ramage, C. A. Barron, S. Bielecki, D. W. Thomas, Tetrahedron Lett. 1987, 4105 |
| Z = OH | |
| Nuc = OH | |
| 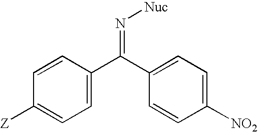 | W. F. DeGrado, E. T. Kaiser, J. Org. Chem. 45 (1980) 1295 |
| Z = Polymer | |
| Nuc = OH | |
| 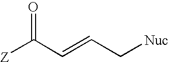 | H. Kunz, B. Dombo, W. Kosch, page 154 and G. Becker, H. Nguyen Trong C. Birr, B. Dombo, H. Kunz, page 157, in each case in Peptides 1988 Proc. 20th Eur. Pept. Symp., deGruyter Berlin 1989 (Editor G. Jung, E. Bayer) |
| Z = OH | |
| Nuc = OH | |
| 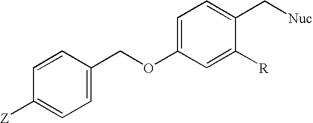 | R = H (Wang linker) R. B. Wang, J. Am. Chem. Soc. 95 (1972) 1328; R = OCH₃ (Sasrin linker) M. Mergler, R. Tanner, J. Gosteli, P. Gross, Tetrahedron Lett. 29 (1988) 4005 |
| Z = Polymer | |
| Nuc = OH | |
| R = H or OCH₃ | |
| Z—CO—CH₂—(p-C₆H₄)—CH₂—Nuc | PAM linker; |
| Z = Polymer | A. R. Mitchell, B. W. Erickson, |
| Nuc = OH | M. N. Raybtsev, R. S. Hodge, |
| | R. B. Merrifield, J. Am. Chem. |
| | Soc. 98 (1976) 7357 |
| 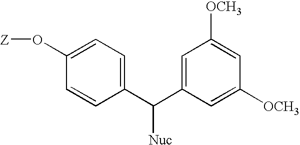 | Rink acid (X = OH) Rink amide (X = NH-Fmoc) H. Rink, Tetrahedron Lett. 1987, 3787 Fmoc = 9-Fluorenyl- methoxy- carbonyl |
| Z = Polymer | |
| Nuc = OH or NH-Fmoc | |
| Z—(p-C₆H₄)—CHNuc-C₆H₅ | BHA linker |
| Z = Polymer | J. Tam, R. D. DiMarchi, |
| Nuc = NH₂ | R. B. Merrifield, Tetrahedron |
| | Lett. 1981, 2851; |
| | A. Hiro, S. Itsuno, J. Hattori, |
| | K. Yamaguchi, S. Nakahama, |
| | N. Yamazaki, J. Chem. Soc. |
| | Chem. Comm. 1983, 25 |
| 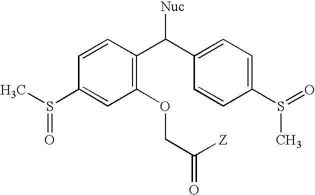 | SCAL (Safety-catch amide linker) M. Patek, M. Lebi, Tetrahedron Lett. 1991, 3891-3894 |
| Z = OH | |
| Nuc = NH₂ | |
| Z—CO—CH₂—p-C₆H₄—CO—CHNuc—CH₃ | F. S. Tjoeng, G. A. Heavner, |
| Z = OH | J. Org. Chem. 48 (1983) 355 |
| Nuc = Cl | |
| Z—CH₂—p-C₆H₄—CO—CHNuc—CH₃ | F. S. Tjoeng, G. A. Heavner, |
| Z= Polymer | J. Org. Chem. 48 (1983) 355 |
| Nuc = Br | |
| 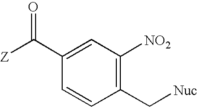 | D. H. Rich, S. K. Gurwara, J. Am. Chem. Soc. 97 (1975) 1575; R. P. Hammer, F. Albericio, E. Grivalt, G. Barany, Int. J. Peptid. Protein Res. 37 (1991) 402; G. Barany, F. Albericio, Peptides Proc. 21st Eur. Pept. Symp. 1991, S. 139; G. Barany, N. A. Sole, R. J. van Abel, F. Alericio, Innovation and Perspectives in Solid Phase Synthesis 1992, pages. 29 and 39 |
| Z = OH | |
| Nuc =Halogen, OH, NH₂ | |
| 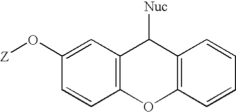 | P. Sieber, Tetrahedron Lett. 1987, 2107 Fmoc = 9-Fluorenyl- methoxy- carbonyl |
| Z = Polymer | |
| Nuc = NH-Fmoc | |
| Z—CO—p-C₆H₄—S—CH₂CH₂—Nuc | Rydon linker |
| Z = OH | P. M. Hardy, H. N. Rydon, |
| Nuc = OH | R. C. Thompson, Tetrahedron |
| | Lett. 1986, 2525-2526 |

The resin polymers which can be used should be insoluble, largely inert to the reaction conditions in stage b) and filterable in the liquid phases which are employed for the reactions and isolation of the compounds; each resin polymer particle preferably has many binding sites for the respective linkers. Depending on the structure of the selected linkers, completely different resin polymers are possible from the synthesis, for example polystyrene resins, polyamide resins, polydimethylacrylamide resins, modified resins based on the resins mentioned and copolymers. Preferred resins are aminomethylenepolystyrene resins, i.e. aminomethylated polystyrene resins, or alternatively differently modified resins based on polystyrene, e.g. graft polymers of polystyrene and polyethylene glycol such as those from the series ®TentaGel (Rapp Polymere, Tübingen, Germany), in the form of swellable particles in a particle size range from, for example 0.01 to 1 mm, preferably 0.05 to 0.5 mm, and a loading of aminomethyl groups from 0.01 to 10 mmol per gram of resin, preferably 0.1 to 2 mmol per gram of resin.

The individual linkers are applied to the resin in a manner known per se; see references mentioned in Table 1. All different sorts of techniques can be employed here. Suitable linker components for the combination with the aminomethylated polystyrene resins are the Raydon linker mentioned and analogous carboxylic acid compounds. Linkers such as the Raydon linker can be reacted under the customary conditions for condensations and especially for amide formation reactions. Gentle methods at moderate temperatures are suitable. The reaction can be carried out, for example, in a largely anhydrous inert organic solvent in the presence of catalysts or customary condensing agents at temperatures from, for example, −30° C. to 200° C., preferably from 0° C. to 150° C., in particular 0° C. to 100° C.

By the designation “inert solvent”, solvents are meant which are inert under the respective reaction conditions, but do not have to be inert under any reaction conditions. For the abovementioned condensation, for example, the following are possible:

ethers such as tert-butyl methyl ether, dimethoxyethane (DME), tetrahydrofuran (THF), diethyl ether, diisopropyl ether,

dipolar aprotic solvents such as dimethylformamide (DMF), N,N-dimethylacetamide (DMA), N-methylpyrrolidone (NMP), acetonitrile,

optionally halogenated aliphatic or aromatic hydrocarbons such as dichloromethane, toluene, o-chlorotoluene, chlorobenzene, or

mixtures of inert solvents.

Suitable condensing means for the preparation of the resin-linker compound (II) from the linker component and an aminomethylenepolystyrene resin are customary means such as azeotropic distillation, reaction with activated derivatives of the respective linker carboxylic acid such as halides or active esters. Gentle methods are particularly suitable, such as reaction in the presence of carbodiimides, such as dicyclohexylcarbodiimide (DCC) or diisopropylcarbodiimide.

The reaction of the acylsulfonamide of the formula (III) with the resin-linker compound (II) to give the resin-linker adduct (IV) takes place according to the invention by a replacement of the nucleofugic group Nuc or of the group Nuc to be activated in the exchange reaction by the amido group of the sulfonylamidocarbonyl group in the compound (III). In the case of a leaving group Nuc=halogen or tosyl etc., the exchange reaction can be carried out with the compound of the formula (III) in the presence of bases, or analogously to the exchange reactions used with the known linkers.

In the case of Nuc=hydroxyl, in particular with linkers such as the Raydon linker, an exchange reaction is preferably carried out under conditions such as are used analogously for a Mitsunobu reaction (Redox reaction), which is known for the condensation of carboxylic acids or other electrophiles with alcohols with the aid of azodicarboxylic acid esters and triphenylphosphane; see, for example, O. Mitsunobu, Synthesis 1981, pages 1-28 and references cited there; also see J. R. Henry et. al., Tetrahedron Lett. 1989, 5709-5712.

The reaction proceeds according to the scheme:

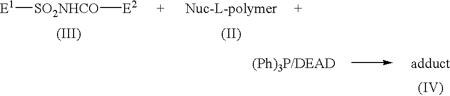

In this case Nuc is a hydroxyl group; Ph is phenyl; DEAD is diethyl azodicarboxylate; other azodicarboxylic acid esters can also be used, such as other reagents and conditions which can be used for carrying out the Mitsunobu reaction. The Mitsunobu reaction used according to the invention can be carried out at moderate temperatures, preferably under anhydrous, neutral conditions at 0° C. to 50° C., in the solvents already mentioned above for the condensation of the linker component to the resin.

Alternatively, other condensation reactions are possible, the particular method depending on the choice of the leaving group Nuc.

The resin-linker adduct (IV) is surprisingly stable, even with respect to the bond between linker L and the nitrogen atom in the acylsulfonamide. The stability makes possible versatile chemical reactions on the radicals E¹ and E². The radicals E¹ and E² must in this case be selected such that a modification of the radicals to give the radicals R¹ or R² in formula (I) is possible.

The potential of the method is illustrated by the following synthesis sequence (cf. Scheme 1):

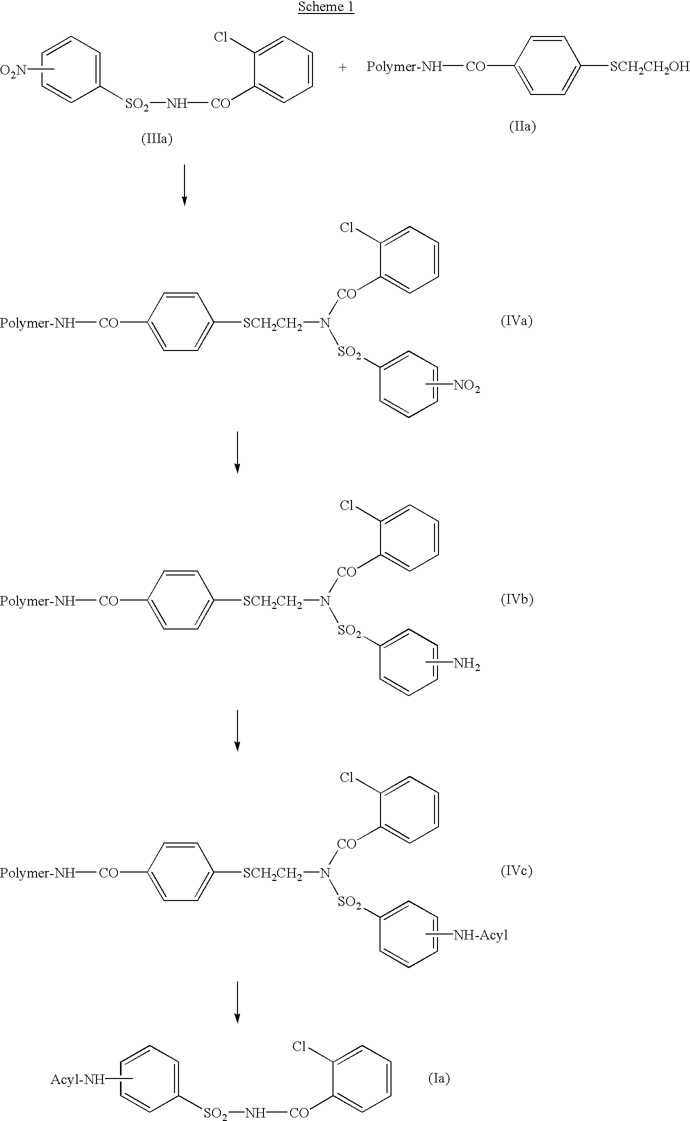

According to Scheme 1, after the binding of the acylsulfonamide of the formula (IIIa) to the resin-linker compound (IIa) the adduct (IVa) is obtained, which can be reduced to the amino group at the nitro group. Many of the chemical reductants suitable for nitro groups are suitable for the reduction, such as metal salts under acidic conditions, preferably mild reductants which can be employed in organic solvents, such as tin dichloride dihydrate/HCl, or catalytic reductions. The amino compound (IVb) obtained can be further modified, e.g. by (reductive) alkylation or by acylation of the amino group to give the compound of the formula (IVc). The acylation can in turn be carried out successfully using a large number of acylating agents, for example using carboxylic acid halides and esters (cf. Scheme 1).

The removal of the compound to be prepared from the resin is carried out by the reaction or reaction sequence typical for the individual linker.

In the case of the Raydon linker, the removal method comprises oxidation of the sulfur atom to the sulfoxide or the sulfone and subsequent β-elimination, the compound of the formula (I) being liberated and an ethenylsulfinyl or ethenylsulfonyl group being formed on the linker (cf. Scheme 1). Suitable oxidants are chemical oxidants for the conversion of thioethers to sulfones, e.g. peroxides such as percarboxylic acids in organic solvents, preferably those such as m-chloroperbenzoic acid in organic solvents. To accelerate the elimination basic conditions are advantageous. For example, the resin particles can be treated after the oxidation step with a basic solution (cleavage solution) which essentially consists of a dipolar aprotic solvent, e.g. ethers such as dioxane, tetrahydrofuran (THF), 1,2-dimethoxyethane or 1,2-diethoxyethane, to which are added small amounts of concentrated sodium hydroxide solution and, if appropriate, further solubilizers, for example from the group consisting of the alcohols, such as methanol, ethanol, propanol, isopropanol or tert-butanol, and ether alcohols, such as methoxyethanol, methoxyethoxyethanol etc. In general, aqueous basic conditions are also suitable, but preferably largely anhydrous conditions using bases in organic solvents.

The preparation of the resin-linker compound and the reactions on it can be carried out using surprisingly many structural variations with respect to the compounds of the formulae (III), (IV) and finally (I). The radicals of the formula E¹ or E² are in general organic radicals, such as have been defined above for R¹ and R².

In this manner, it is possible to prepare and to modify active compound structures in a controlled and standardized manner. Some of the compounds of the formula (Ia) and their use as safeners have already been proposed in the German Patent Application No. 19621522.6. By variation of the starting compounds of the formula (III) and the derivatization reactions, the preparation method according to the invention thus makes possible a rapid, systematic preparation of structurally variant compounds, e.g. in the area of the group of safeners mentioned, which can reduce or suppress phytotoxic side effects of herbicides, or in the area of other chemical compounds, especially of the active compounds for use in human medicine, veterinary medicine or in plant protection.

Preferred radicals for the definitions of R¹, R², E¹, E², (E¹)N and (E²)N are: aliphatic or aromatic hydrocarbon radicals or hydrocarbon-oxy radicals such as alkyl, alkoxy, alkenyl, alkenyloxy, alkynyl, alkynyloxy, cycloalkyl, cycloalkoxy, aryl, preferably phenyl, or aryloxy or heterocyclic radicals, where each of the above radicals is in each case unsubstituted or substituted by one or more radicals from the group consisting of halogen, hydroxyl, amino, mono- and disubstituted amino, nitro, cyano, cyanato, thiocyanato, azido, alkyl, cycloalkyl, alkenyl, cycloalkenyl, alkynyl, alkoxy, alkylthio, alkenyloxy, cycloalkoxy, cycloalkenyloxy and alkynyloxy,

where each of the last-mentioned 15 radicals is unsubstituted or substituted by one or more radicals from the group consisting of halogen, CN, N0₂, alkoxy, alkylthio, haloalkoxy, acyl, acyloxy, amino, mono- and disubstituted amino and, in the case of cyclic radicals, also alkyl and haloalkyl,

and unsubstituted and substituted aryl, unsubstituted and substituted aryloxy, unsubstituted and substituted heterocyclyl, unsubstituted and substituted heterocyclyloxy, acyl and acyloxy.

Furthermore of interest are processes according to the invention for the preparation of compounds of formula (I), in which

R¹ is (C₁-C₆)alkyl, (C₃-C₆)cycloalkyl, (C₂-C₆)alkenyl, (C₅-C₆)cycloalkenyl or (C₂-C₆)alkynyl, where each of the last-mentioned 5 radicals is unsubstituted or substituted by one or more radicals from the group consisting of halogen, (C₃-C₆)cycloalkyl, (C₁-C₄)alkoxy, (C₁-C₄)alkylthio, (C₁-C₄)haloalkoxy, unsubstituted and substituted aryl, unsubstituted and substituted heterocyclyl, unsubstituted and substituted aryloxy and, in the case of cyclic radicals, also (C₁-C₄)alkyl and (C₁-C₄)haloalkyl, or is phenyl or heterocyclyl, where each of the two last-mentioned radicals is unsubstituted or substituted by one or more radicals from the group consisting of halogen, nitro, cyano, thiocyanato, amino, mono- and disubstituted amino, (C₁-C₄)alkyl, (C₁-C₄)alkoxy, (C₁-C₄)alkylthio, (C₁-C₄)alkylsulfinyl, (C₁-C₄)alkylsulfonyl, (C₁-C₅)alkanoyl, (C₁-C₄)alkoxycarbonyl and phenylcarbonyl, where each of the last-mentioned 8 radicals is unsubstituted or substituted by one or more radicals from the group consisting of halogen, (C₁-C₄)alkoxy, (C₁-C₄)alkylthio, (C₁-C₄)haloalkoxy and, in the case of cyclic radicals, also (C₁-C₄)alkyl and (C₁-C₄)haloalkyl, and

R² is (C₁-C₆)alkyl, (C₃-C₆)cycloalkyl, (C₂-C₆)alkenyl, (C₅-C₆)cycloalkenyl, (C₂-C₆)alkynyl, (C₁-C₆)alkoxy, (C₁-C₆)alkylthio, (C₃-C₆)alkenyloxy, (C₃-C₆)cycloalkoxy, (C₅-C₆)cycloalkenyloxy or (C₃-C₆)alkynyloxy, where each of the last-mentioned 11 radicals is unsubstituted or substituted by one or more radicals from the group consisting of halogen, (C₃-C₆)cycloalkyl, (C₁-C₄)alkoxy, (C₁-C₄)alkylthio, (C₁-C₄)haloalkoxy, unsubstituted and substituted aryl, unsubstituted and substituted heterocyclyl, unsubstituted and substituted aryloxy and, in the case of cyclic radicals, also (C₁-C₄)alkyl and (C₁-C₄)haloalkyl, or is

phenyl, phenoxy, heteroaryl or heteroaryloxy, where each of the four last-mentioned radicals is unsubstituted or substituted by one or more radicals from the group consisting of halogen, nitro, cyano, thiocyanato, amino, mono- and disubstituted amino, (C₁-C₄)alkyl, (C₁-C₄)alkoxy, (C₁-C₄)alkylthio, (C₁-C₄)alkylsulfinyl, (C₁-C₄)alkylsulfonyl, (C₁-C₅)alkanoyl, (C₁-C₄)alkoxycarbonyl and phenylcarbonyl, where each of the last-mentioned 8 radicals is unsubstituted or substituted by one or more radicals from the group consisting of halogen, (C₁-C₄)alkoxy, (C₁-C₄)alkylthio, (C₁-C₄)haloalkoxy and in the case of cyclic radicals, also (C₁-C₄)alkyl and (C₁-C₄)haloalkyl.

Furthermore preferred are preparations according to the invention to give compounds of the formula (I), in which

R¹ is (C₁-C₆)alkyl which is unsubstituted or substituted by one or more radicals from the group consisting of halogen, (C₃-C₆)cycloalkyl, (C₁-C₄)alkoxy, (C₁-C₄)alkylthio, (C₁-C₄)haloalkoxy, phenyl and heteroaryl, where each of the two last-mentioned radicals is unsubstituted or substituted by one or more radicals from the group consisting of halogen, nitro, cyano, (C₁-C₄)alkyl, (C₁-C₄)haloalkyl, (C₁-C₄)alkoxy, (C₁-C₄)alkylthio and (C₁-C₄)haloalkoxy, or is (C₃-C₆)cycloalkyl which is unsubstituted or substituted by one or more radicals from the group consisting of halogen, (C₁-C₄)alkyl, (C₁-C₄)haloalkyl, (C₁-C₄)alkoxy, (C₁-C₄)alkylthio and (C₁-C₄)haloalkoxy, or is

phenyl or heteroaryl, where each of the two last-mentioned radicals is unsubstituted or substituted by one or more radicals from the group consisting of halogen, nitro, cyano, thiocyanato, amino, mono- and di[(C₁-C₄)alkyl]amino, acylamino, N-acyl-N-(C₁-C₄)alkylamino, (C₁-C₄)alkyl, (C₁-C₄)alkoxy, (C₁-C₄)alkylthio, (C₁-C₄)alkylsulfinyl, (C₁-C₄)alkylsulfonyl, (C₁-C₅)alkanoyl, (C₁-C₄)alkoxycarbonyl and phenylcarbonyl, where each of the last-mentioned 8 radicals is unsubstituted or substituted by one or more radicals from the group consisting of halogen, (C₁-C₄)alkoxy, (C₁-C₄)alkylthio, (C₁-C₄)haloalkoxy and, in the case of cyclic radicals, also (C₁-C₄)alkyl and (C₁-C₄)haloalkyl, and

R² is (C₁-C₆)alkyl or (C₁-C₆)alkoxy, where each of the two last-mentioned radicals is unsubstituted or substituted by one or more radicals from the group consisting of halogen, (C₃-C₆)cycloalkyl, (C₁-C₄)alkoxy, (C₁-C₄)alkylthio, (C₁-C₄)haloalkoxy, phenyl, phenoxy and heteroaryl, where each of the three last-mentioned radicals is unsubstituted or substituted by one or more radicals from the group consisting of halogen, nitro, cyano, (C₁-C₄)alkyl, (C₁-C₄)haloalkyl, (C₁-C₄)alkoxy, (C₁-C₄)alkylthio and (C₁-C₄)haloalkoxy, or is (C₃-C₆)cycloalkyl, (C₂-C₆)alkenyl, (C₂-C₆)alkynyl, (C₁-C₆)alkylthio, (C₃-C₆)alkenyloxy, (C₃-C₆)cycloalkoxy or (C₃-C₆)alkynyloxy or phenyl, phenoxy, heteroaryl or heteroaryloxy, where each of the four last-mentioned radicals is unsubstituted or substituted by one or more radicals from the group consisting of halogen, nitro, cyano, thiocyanato, amino, mono- and di[(C₁-C₄)alkyl]amino, acylamino, N-acyl-N-(C₁-C₄)alkylamino, (C₁-C₄)alkyl, (C₁-C₄)alkoxy, (C₁-C₄)alkylthio, (C₁-C₄)alkylsulfinyl, (C₁-C₄)alkylsulfonyl, (C₁-C₅)alkanoyl, (C₁-C₄)alkoxycarbonyl and phenylcarbonyl, where each of the last-mentioned 8 radicals is unsubstituted or substituted by one or more radicals from the group consisting of halogen, (C₁-C₄)alkoxy, (C₁-C₄)alkylthio and (C₁-C₄)haloalkoxy and, in the case of cyclic radicals, also (C₁-C₄)alkyl and (C₁-C₄)haloalkyl.

Particularly preferred are preparations according to the invention to give compounds of the formula (I), in which

R¹ is (C₁-C₄)alkyl, (C₁-C₄)haloalkyl, (C₃-C₆)cycloalkyl, which is unsubstituted or substituted by one or more radicals from the group consisting of halogen, (C₁-C₄)alkyl and (C₁-C₄)haloalkyl, or is

phenyl which is unsubstituted or substituted by one or more radicals from the group consisting of halogen, nitro, cyano, amino, mono- and di[(C₁-C₄)alkyl]amino, acylamino, N-acyl-N-(C₁-C₄)alkylamino, (C₁-C₄)alkyl, (C₁-C₄)alkoxy and (C₁-C₄)alkylthio, and

R² is (C₁-C₄)alkyl or (C₁-C₄)alkoxy, where each of the two last-mentioned radicals is unsubstituted or substituted by one or more radicals from the group consisting of halogen, (C₃-C₆)cycloalkyl, (C₁-C₄)alkoxy, (C₁-C₄)alkylthio and (C₁-C₄)haloalkoxy, or is

phenyl which is unsubstituted or substituted by one or more radicals from the group consisting of halogen, nitro, cyano, amino, mono- and di[(C₁-C₄)alkyl]amino, acylamino, N-acyl-N-(C₁-C₄)alkylamino, (C₁-C₄)alkyl, (C₁-C₄)alkoxy, (C₁-C₄)alkylthio, (C₁-C₄)alkylsulfinyl, (C₁-C₄)alkylsulfonyl, (C₁-C₅)alkanoyl, (C₁-C₄)alkoxycarbonyl and phenylcarbonyl.

Furthermore particularly preferred are preparations according to the invention to give compounds of the formula (I), in which

R¹ is phenyl which is unsubstituted or substituted by a radical from the group consisting of nitro, amino, mono- and di[(C₁-C₄)alkyl]amino, (C₁-C₄)alkylcarbonylamino, (C₁-C₄)alkoxycarbonylamino, mono- and di[(C₁-C₄)alkyl]aminocarbonylamino, and

R² is (C₁-C₄)alkoxy or

phenyl which is unsubstituted or substituted by one or more radicals from the group consisting of halogen, nitro, cyano, (C₁-C₄)alkyl, (C₁-C₄)alkoxy and (C₁-C₄)alkylthio.

Compounds of the formula (IV), (IV′) and (IV″) with corresponding radicals or correspondingly suitable precursors of the radicals correspond to the compounds of the formula (I) with the radicals R¹ and R².

Many of the prepared and the preparable compounds of the formula (I) are novel. In particular, novel compounds of the formula (I) are those in which

R¹ is phenyl which is substituted in the meta or ortho position by a radical of the formula mono- or di[(C₁-C₄)alkyl]amino, (C₁-C₄)alkylcarbonylamino, (C₁-C₄)alkoxycarbonylamino, mono- or di[(C₁-C₄)alkyl]aminocarbonylamino, and

R² is (C₁-C₄)alkoxy or

phenyl which is unsubstituted or substituted by one or more radicals from the group consisting of halogen, nitro, cyano, (C₁-C₄)alkyl, (C₁-C₄)alkoxy and (C₁-C₄)alkylthio.

These compounds are accessible in a systematic manner by the process according to the invention and are suitable in some cases as active compounds for plant protection and as useful intermediates for the preparation of biologically active compounds.

In the following examples, quantitative data relates to the weight, if not stated otherwise. Abbreviations used in the examples are familiar to a person skilled in the art or are in some cases explained at another place in the description.

### EXAMPLES

1. Preparation of the Resin-Linker Compound

1a) Preparation of the Linker 4-(2-hydroxyethylthio)benzoic Acid

2.0 l of methylene chloride were treated under a nitrogen atmosphere with 46.92 g (304 mmol) of 4-mercaptobenzoic acid. 57.0 g (456 mmol) of 2-bromoethanol and 61.5 g (609 mmol) of triethylamine were added dropwise under a nitrogen atmosphere. The solution was stirred overnight at room temperature. A little undissolved solid was then filtered off the filtrate was concentrated and the residue was taken up in 500 ml of 2N NaOH. This phase was extracted once with ether. The aqueous phase was then acidified to pH=4 and the precipitated product was isolated by filtration. After crystallization from acetonitrile, the product defined under title 1a) was obtained; yield: 34.21 g (56.7%); m.p.: 150° C.

1b) Preparation of the Resin-Linker Compound N-(4-hydroxyethylthiobenzoyl)aminomethylenepolystyrene Resin

20.0 g of aminomethylenepolystyrene resin (1.07 mmol of amino function per gram of resin) were suspended in 200 ml of anhydrous tetrahydrofuran (THF) with 6.36 g (32.1 mmol) of 4-(hydroxyethylthio)benzoic acid and 2.89 g (21.4 mmol) of 1-hydroxybenzotriazole. 8.10 g (64.2 mmol) of diisopropylcarbodiimide were added under a nitrogen atmosphere. The suspension stood at room temperature for 64 h and was then filtered. The product was washed five times with 100 ml of dimethylformamide (DMF) and methanol each time, then several times with THF and finally with ether. The resin obtained was dried in a desiccator. The resin-linker adduct defined under the title 1b) was obtained with a crude yield of 24.8 g (104% of theory).

2. Preparation of the Resin-Linker Adduct with a Sulfonamide

2a) Preparation of N-(2-chlorobenzoyl)-3-nitrophenylsulfonamide

20.0 g (98.9 mmol) of 3-nitrophenylsulfonamide were initially introduced into 300 ml of anhydrous acetonitrile together with 243 mg (1.98 mmol) of 4-N,N-dimethylaminopyridine. After addition of 23.0 g (228 mmol) of triethylamine, the mixture was cooled to 0° C. and 19.04 g (108.8 mmol) of 2-chlorobenzoyl chloride were added dropwise. After removal of the cooling, the reaction solution warmed to room temperature in the course of 4 h. The solution was concentrated in vacuo and the residue was taken up in ethyl acetate. The organic phase was extracted twice with 2N HCl, washed with water and dried over magnesium sulfate. After filtering off the magnesium sulfate and removing the solvent, the crude product was crystallized from toluene. N-(2-Chlorobenzoyl)-3-nitrophenylsulfonamide was obtained in a yield of 31.2 g (92.6%); m.p.:135° C.

2b) Preparation of the Adduct N-{4-[N-(2-chlorobenzoyl)-N-(3-nitrophenylsulfonyl)aminoethylthio]-benzoyl}aminomethylenepolystyrene Resin

10.0 g (8.97 mmol of hydroxyl function) of N-(4-hydroxyethylthiobenzoyl)-aminomethylenepolystyrene resin was suspended in a solution of 9.17 g (26.9 mmol) of N-(2-chlorobenzoyl)-3-nitrophenylsulfonamide and 9.40 g (35.9 mmol) of triphenylphosphane. 3.96 g (31.4 mmol) of diethyl azodicarboxylate were added under a nitrogen atmosphere and the mixture was stirred at room temperature for 6 h. For working up, it was filtered and washed three times with 100 ml of dimethylformamide, THF and diethyl ether. The resin obtained, defined under title 2b) was dried overnight in a desiccator; crude yield: 13.62 g (105.6% of theory).

3. Reactions with the Resin-Linker Adduct

3.1 Reduction of the Nitro Group to Give the Product N{4-[N-(2-chloro-benzoyl)-N-(3-aminophenylsulfonyl)aminoethylthio]benzoyl}-aminomethylenepolystyrene Resin

1.57 g (6.96 mmol) of tin dichloride dihydrate were dissolved in 45 ml of dimethylformamide and treated with 2.5 ml of concentrated hydrochloric acid. 1.00 g (0.70 mmol of nitro function) of N-{4-[N-(2-chlorobenzoyl)-N-(3-nitrophenylsulfonyl)aminoethylthio]benzoyl}aminomethylenepolystyrene resin was added and the mixture was stirred at 50-55° C. for 4 h, then cooled and filtered. The resin obtained was washed three times each with 20 ml each time of dimethylformamide, THF, dioxane, methylene chloride and finally with diethyl ether. After drying the product, the title compound (3.1) was obtained with crude yield of 1.05 g (107.2% of theory).

3.2 Acylation of the Amino Function and Removal to Give N-(2-chloro-benzoyl)-3-(isopropylcarbonylamino)benzenesulfonamide

0.300 g (0.210 mmol of amino function) of N-{4-[N-(2-chlorobenzoyl)-N-(3-aminophenylsulfonyl)aminoethylthio]benzoyl}aminomethylenepolystyrene resin were suspended in 30 ml of methylene chloride, cooled to −10° C. under a nitrogen atmosphere and treated with 227 mg (2.13 mmol) of isopropylcarbonyl chloride. 26 mg (0.21 mmol) of 4-N,N-dimethylaminopyridine and 215 mg (2.13 mmol) of triethylamine were then added with stirring. The suspension stood overnight at room temperature and was then filtered. The acylated resin obtained was washed three times each with 20 ml each time of methylene chloride, dimethylformamide, THF and diethyl ether. The acylated resin was finally suspended in 10 ml of methylene chloride and oxidized for 10 min using 410 mg (1.66 mmol) of 70% strength 3-chloroperbenzoic acid. The suspension was filtered and washed three times each with 20 ml of methylene chloride and dioxane. After this, 4 ml of cleavage solution were added (cleavage solution: 25 ml of dioxane, 3 ml of 2-methoxyethanol, 0.5 ml of 5N NaOH in H₂O) and the mixture was allowed to react for 10 min. The solution was filtered off and the process was repeated. The combined filtrates were filtered through 1.5 g of acidic alumina. The alumina was washed twice with a solution of dioxane/ethanol (7:3). The combined filtrates were concentrated to dryness and 79.1 mg (92%) of the desired title product were obtained; m.p. 177° C. (dec.); ¹H-NMR (DMSO-d₆, TMS): δ=1.12 (d, J=5.5 Hz, 6H, CH—(CH₃)₂), 2.64 (sept., J=5.5 Hz, 1H, CH—(CH₃)₂), 7.35-7.65 (m, 6H, aromatic H), 7.90 (m, 1H, aromatic H), 8.40 (m, 1H, aromatic H), 10.20 (s, 1H, NH—CO—CH), 12.40 (s, 1H, SO₂—NH—CO).

4. Preparation of the Sulfonamide N-methoxycarbonyl-3-nitrophenyl-sulfonamide

4.40 g (108 mmol) of sodium hydride (60% strength in oil) were suspended in 250 ml of anhydrous THF. The suspension was treated first with a solution of 10.0 g (49.5 mmol) of 3-nitrophenylsulfonamide in 100 ml of anhydrous tetrahydrofuran and then with a solution of 5.60 g (54.4 mmol) of methyl chloroformate. After stirring at room temperature for 100 h, the mixture was added to ice water, acidified with 2 N HCl and washed three times with ethyl acetate, then dried over sodium sulfate, filtered and concentrated. The crude product was crystallized from ethyl acetate/n-heptane and N-methoxycarbonyl-3-nitrophenylsulfonamide was obtained with a yield of 8.64 g (67%); m.p.: 121-123° C.

5. Preparation of the Resin-Linker Adduct

N-{4-[N-(methoxycarbonyl)-N-(3-nitrophenylsulfonyl)-aminoethylthio]benzoyl}aminomethylenepolystyrene Resin

3.00 g (2.69 mmol of hydroxyl function) of 4-hydroxyethylthio-benzoylaminomethylenepolystyrene resin were suspended in 100 ml of THF under an N₂ atmosphere and then treated with 2.10 g (8.07 mmol) of N-methoxycarbonyl-3-nitrobenesulfonamide and 2.82 g (10.8 mmol) of triphenylphosphane. 1.19 g (9.42 mmol) of diethyl azodicarboxylate were added dropwise to this suspension. After allowing it to stand at room temperature for 16 h, the resin was filtered off and washed three times each with 50 ml each time of dimethylformamide, tetrahydrofuran and diethyl ether. After drying in a desiccator, the title product (5.) was obtained in a crude yield of 3.89 g (107%).

6. Reduction of the Nitro Group on the Resin-Linker Adduct to Give the Product N-{4-[N-(methoxycarbonyl)-N-(3-aminophenylsulfonyl)-aminoethylthio]benzoyl}aminomethylenepolystyrene Resin

3.59 g (26.5 mmol) of N-{4-[N-(methoxycarbonyl)-N-(3-nitrophenylsulfonyl)-aminoethylthio]benzoyl}aminomethylenepolystyrene resin were warmed to 55° C. for 4 h in a solution of 5.95 g (26.5 mmol) of tin dichloride dihydrate and 4.20 ml of concentrated hydrochloric acid in 40 ml of dimethylformamide. The resin was filtered off and washed three times each with 20 ml of dimethylformamide, THF and ether. After drying in a desiccator, the crude product of the title compound (6.) was obtained; crude yield: 3.63 g (103%).

7. Acylation and Cleavage to Give N-(methoxycarbonyl)-3-(isopropyl-carbonylamino)benzenesulfonamide

300 mg (230 pmol of amino function) of N{4-[N-(methoxycarbonyl)-N-(3-aminophenylsulfonyl)aminoethylthio]benzoyl}aminomethylenepolystyrene resin were suspended in 6 ml of methylene chloride under a nitrogen atmosphere, cooled to 0° C. and treated with 241 mg (2.26 mmol) of isopropylcarbonyl chloride. 28 mg (0.23 mmol) of 4-N,N′-dimethylamino-pyridine and 229 mg (2.26 mmol) of triethylamine were then added with stirring. The suspension stood overnight at room temperature and was then filtered. The resin was washed with 25 ml each of methylene chloride, dimethylformamide, THF and ether. Following this, the acylated resin was suspended in 4 ml of methylene chloride and oxidized for 10 min using 410 mg (1.66 mmol) of 70% strength 3-chloroperbenzoic acid. The suspension was filtered again and washed three times each with 4 ml of methylene chloride and dioxane. 4 ml of a cleavage solution were then added (cleavage solution: 25 ml of dioxane, 3 ml of 2-methoxyethanol, 0.5 ml of 5N NaOH in H₂O) and the mixture was allowed to react for 10 min. The cleavage solution was filtered off and the process was repeated. The combined filtrates were filtered through 1.5 g of acidic alumina. The alumina was washed with dioxane/ethanol (7:3). The combined filtrates were concentrated to dryness and N-(methoxycarbonyl)-3-(isopropylcarbonylamino)benzenesulfonamide was obtained with a yield of 67.4 mg (97.6% of theory);¹H-NMR (DMSO-d₆, TMS): δ=1.10 (d, J=6.0 Hz, 6H, CH—(CH₃)₂), 2.65 (sept., J=6.0 Hz, 1H, CH—(CH₃)₂), 3.55 (s, 3H, O—CH₃), 7.48 (m, 2H, aromatic H), 7.85 (m, 1H, aromatic H), 8.20 (m, 1H, aromatic H), 10.20 (s, 1H, NH—CO—CH), 12.20 (s, 1H, SO₂—NH—CO).

The compounds shown in Table 2 which follows were prepared in an analogous manner to Examples 1 to 7.

TABLE 2
Further resin-linker adducts of the formula (IV)

| | |
|---|---|
| | (IV) |
| 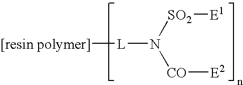 | |

resin polymer = aminomethylene resin;
L = —CO—p-C₆H₄—S—CH₂CH₂—
E¹ = —C₆H₄—R^{a}

| R^{a} | E² | (R^{b})ₘ |
|---|---|---|
| 4-NO₂ | 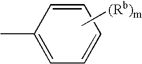 | 2-OCH₃ |
| 4-NO₂ | ″ | 2-I |
| 4-NO₂ | ″ | 3-NO₂ |
| 3-NO₂ | ″ | 2-OCH₃ |
| 3-NO₂ | ″ | 2-I |
| 3-NO₂ | ″ | 4-I |
| 4-J | ″ | 2-OCH₃ |
| 4-J | ″ | 3-NO₂ |
| 4-J | ″ | 2-NO₂ |
| 3-NO₂ | ″ | 2,4-(CH₃)₂ |
| 3-NO₂ | ″ | 2,5-(CH₃)₂ |
| 4-NO₂ | ″ | 2,4-(CH₃)₂ |
| 4-NO₂ | ″ | 2,5-(CH₃)₂ |
| 3-NO₂ | ″ | 2-CH₃-4-Cl |
| 3-NO₂ | ″ | 2-CH₃-5-Cl |
| 4-NO₂ | ″ | 2-CH₃-4-Cl |
| 4-NO₂ | ″ | 2-CH₃-5-Cl |
| 3-NO₂ | ″ | 2,4,5-F₃ |
| 4-NO₂ | ″ | 2,4,5-F₃ |
| 3-NO₂ | ″ | 2,6-(OCH₃)₂ |
| 4-NO₂ | ″ | 2,6-(OCH₃)₂ |
| 3-NO₂ | ″ | 3-OCF₃ |
| 4-NO₂ | ″ | 3-OCF₃ |
| 3-NO₂ | naphth-1-yl | |
| 4-NO₂ | ″ | |
| 3-NO₂ | 1-methoxynaphth-2-yl | |
| 4-NO₂ | ″ | |
| 3-NO₂ | 3-methoxynaphth-2-yl | |
| 4-NO₂ | ″ | |

The preparation of the compounds of the formula (IV) (binding) took place in yields of 85-95% of theory and purities of over 95%. The cleavages of the bound acylsulfonamides by oxidation with m-chloroperbenzoic acid and β-elimination analogously to the method in Examples 3.2 and 7 likewise took place in high yields of over 90% of theory and purities of more than 95%.

### Example 8

Corresponding to the above Examples 1 to 7 and Scheme 1, starting from the compound N-(3-methoxynaphthalen-2-ylcarbonyl)-4-nitrophenyl-sulfonamide of the formula (IIIa-N) [see Scheme 2] the resin-linker adduct (IVa-N) was prepared by reacting with the resin-linker compound from Example 1b) analogously to Example 2b). The reduction of the nitro group in adduct (IVa) analogously to Example 3.1 affords compound (IVb-N), which is acylated with carbonyl chlorides from Table 3 analogously to Example 3.2 to give corresponding resin-linker adducts of the formula (IVc-N). The subsequent cleavage from the resin (cf. also Example 3.2) affords the respective sulfonamides of the formula (Ia-N) [see Scheme 2 in combination with Table 3].

In this manner, in a series 45 sulfonamides (Ia-N) were obtained in purities of 45 to 100% and in each case in an amount which was sufficient for the individual tests for biological properties (greenhouse screening).

The structures of the individual sulfonamides (1a-N) were confirmed by comparison (HPLC, TLC) with sulfonamides prepared in a conventional manner or by customary methods of structural elucidation (e.g. elemental analysis, ¹H-NMR, IR, MS).

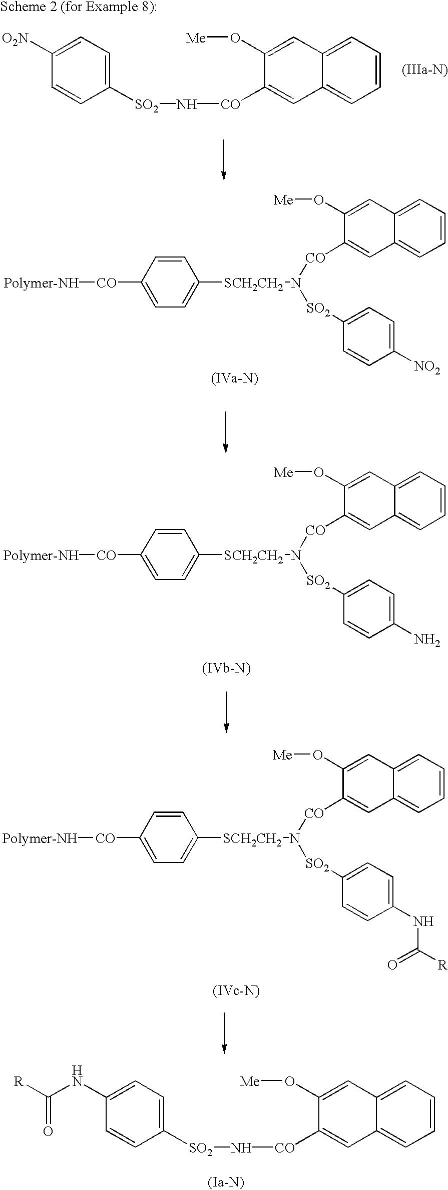

In Scheme 2 and Table 3 which follows, “Me” is in each case methyl.

TABLE 3

| | |
|---|---|
| Acid chlorides of the formula (1) for the reaction with resin-linker | (1) |
| adduct (IVb-N) | |
| R—CO—Cl | |

| Cpd. No. | R |
|---|---|
| 1 | —CH(CH₃)CH₂CH₃ |
| 2 | cyclohexyl |
| 3 | adamant-1-yl |
| 4 | —C(CH₃)₂O—CO—CH₃ |
| 5 | —CH(C₆H₅)₂ |
| 6 | thien-2-ylmethyl |
| 7 | naphth-1-yl |
| 8 | 3,4-dimethoxybenzyl |
| 9 | pent-1-yl |
| 10 | —CH₂—O—CH₃ |
| 11 | cycloprop-1-yl |
| 12 | benzyl |
| 13 | n-prop-1-yl |
| 14 | n-hept-1-yl |
| 15 | —CH₂—O—C₆H₄-p-Me |
| 16 | —(CH₂)₁₆—CH₃ |
| 17 | furan-2-yl |
| 18 | tert-butyl |
| 19 | —(CH₃)₁₅—CH₃ |
| 20 | ethyl |
| 21 | CH₂—O—CH₂—C₆H₅ |
| 22 | CH(C₂H₅)—O—C₆H₅ |
| 23 | CH(CH₃)—O—C₆H₅ |
| 24 | CH(C₂H₅)-n-C₄H₉ |
| 25 | CCl₃ |
| 26 | 3,4-dichlorophenyl |
| 27 | 4-trifluoromethylphenyl |
| 28 | 2-fluorophenyl |
| 29 | 4-ethylphenyl |
| 30 | 4-methylphenyl |
| 31 | 4-fluorophenyl |
| 32 | 2,4-difluorophenyl |
| 33 | 3-trifluoromethoxyphenyl |
| 34 | 3-chlorophenyl |
| 35 | 2-chlorophenyl |
| 36 | 3,5-dichlorophenyl |
| 37 | 2-bromophenyl |
| 38 | 2,6-difluorophenyl |
| 39 | 2,6-dichlorophenyl |
| 40 | 2,4,5-trifluorophenyl |
| 41 | 4-bromophenyl |
| 42 | phenyl |
| 43 | thien-2-yl |
| 44 | methyl |
| 45 | isopropyl |

1. A process for the preparation of chemical compounds of the formula (I)
R¹—SO₂—NH—CO—R² (I)
in which R¹ and R² are each an organic radical,
which comprises
a) reacting a resin-linker compound of the formula (II)
[resin polymer]-[L-Nuc]ₙ (II)
in which
[resin polymer] is the radical of a resin which is connected via n binding sites with the n groups of the formula -L-Nuc as defined in formula (II),
L is in each case an organic linker,
Nuc is a nucleofugic group (leaving group) or a group to be activated under the reaction conditions to give a leaving group,
n is the number of functional groups L-Nuc on the resin and is greater than or equal to 1,
with an acylsulfonamide of the formula (III)
E¹-SO₂—NH—CO—E² (III)
in which E¹ and E² independently of one another in each case are an organic radical which is suitable for the preparation of the radicals R¹ and R² in compound (I),
in the presence of a condensing agent to give a resin-bound adduct of the formula (IV) 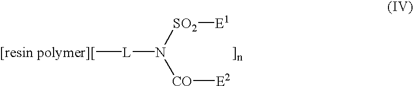
in which [resin polymer], L, n, E¹ and E² are as defined in formula (II) or formula (III),
b) derivatizing the adduct (IV) obtained in one or more further reaction steps on the organic radicals E¹ or E² and thus optionally reacting via resin-bound intermediates of the formula (IV′), which in contrast to formula (IV) contain the organic radicals (E¹)′ or (E²)′ of the derivatives, to give the compound (IV″) 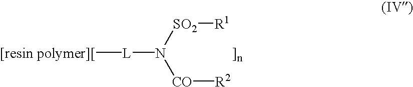
in which R¹ and R² are as defined in formula (I) and [resin polymer], L and n are as defined in formula (II) or formula (IV), and
c) removing the compound of the formula (I) from the resin-linker adduct of the formula (IV″).

2. The process as claimed in claim 1, wherein the linker is a linker from the group of linkers which can be employed for the binding of carboxylic acids in resin-bound synthesis.

3. The process as claimed in claim 1, wherein in formula (II) the linker L-Nuc is a group of the formula —CO-p-C₆H₄—S—CH₂CH₂—OH and the compound (II) is reacted with the acylsulfonamide as acidic component to give the resin-linker adduct of the formula (IV) under the condensing conditions as are analogously used for carboxylic acids according to the Mitsunobu reaction.

4. The process as claimed in claim 3, wherein the cleavage of the compound of the formula (I) from the compound of the formula (IV″) is carried out with oxidation of the sulfur atom in the linker L to the sulfone and subsequent β-elimination.

5. A compound of the formula (IV) 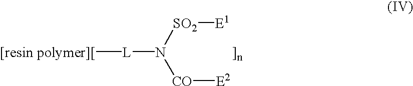
in which
[resin polymer] is the radical of a resin which is connected to the groups of the formula E¹-SO₂—N—CO-E² via n binding sites and the linkers of the formula L,
L is in each case an organic linker,
n is the number of the groups bonded to the resin via the linker L,
E¹, E² independently of one another in each case are an organic radical.

6. A process for the preparation of a compound of the formula (IV) as defined in claim 5, which comprises reacting a resin-linker compound of the formula (II)
[resin polymer]-[L-Nuc]ₙ (II)
in which
[resin polymer], L and n are as defined in formula (IV) and
Nuc is a nucleofugic group (leaving group) or group to be activated under the reaction conditions to give a leaving group,
with an acylsulfonamide of the formula (III)
E¹-SO₂—NH—CO-E² (III)
in which E¹ and E² are as defined in formula (IV),
in the presence of a condensing agent to give the resin-bound adduct of the formula (IV).

7. A process as claimed in claim 6, wherein the linker L-Nuc in formula (II) is a group of the formula —CO-p-C₆H₄—CH₂CH₂—OH and the compound (II) is reacted with the acylsulfonamide as acidic component to give the resin-linker adduct of the formula (IV) under the analogous conditions for a Mitsunobu reaction.

8. A process for the preparation of a compound of the formula (IV″) 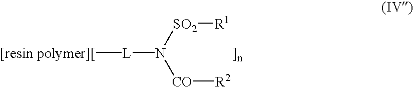
in which [resin polymer], L, n, R¹, R² are as defined in formulae (I) and (II) in claim 1, which comprises derivatizing a compound of the formula (IV) 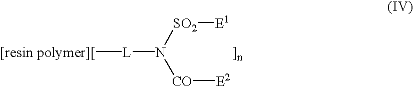
in which
[resin polymer], L and n are as defined in formula (IV″) and
E¹, E² independently of one another in each case are an organic radical suitable for the preparation of the radicals R¹ or R² in compound (IV″),
in one or more further reaction steps on the organic radicals E¹ or E² and thus optionally reacting via resin-bonded intermediates of the formula (IV), which in contrast to formula (IV) contain the organic radicals (E¹′) or (E²′) of the derivatives, to give the compound (IV″).

9. A process for the preparation of a compound of the formula (I),
R¹—SO₂—NH—CO—R² (I)
in which R¹ and R² in each case are an organic radical,
which comprises cleaving the compound of the formula (I) from the resin-linker adduct of the formula (IV″) 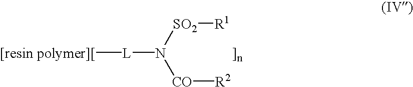
in which R¹ and R² are as defined in formula (I) and [resin polymer], L and n are as defined in formula (II) or formula (IV″) as in claim 1.

10. The process as claimed in claim 9, wherein the linker L in formula (IV″) is a group of the formula —CO-p-C₆H₄—S—CH₂CH₂—, the carbonyl group of the linker being bonded to the resin polymer, and the cleavage of the compound of the formula (I) from the compound of the formula (IV″) taking place with oxidation of the sulfur atom in the linker L to the sulfone and subsequent β-elimination.

11. A compound of the formula (I)
R¹—SO₂—NH—CO—R² (I)
in which
R¹ is phenyl which is substituted in the meta or ortho position by a radical of the formula mono- or di[(C₁-C₄)alkyl]amino, (C₁-C₄)alkylcarbonylamino, (C₁-C₄)alkoxycarbonylamino, mono- or di[(C₁-C₄)alkyl]aminocarbonylamino, and
R² is (C₁-C₄)alkoxy or
phenyl which is unsubstituted or substituted by one or more radicals from the group consisting of halogen, nitro, cyano, (C₁-C₄)alkyl, (C₁-C₄)alkoxy and (C₁-C₄)alkylthio.
